(19) United States
(12) Patent Application Publication
(10) Pub. No.: US 2012/0232467 A1
(43) Pub. Date: Sep. 13, 2012

(54) OXYGEN BATH SYSTEM

(75) Inventor: Shoichi Nakamura, Higashichikuma-gun (JP)

(73) Assignees: ACP JAPAN CO., LTD., Tokyo (JP); Shoichi Nakamura, Higashichikuma-gun, Nagano (JP)

(21) Appl. No.: 13/511,283

(22) PCT Filed: Dec. 27, 2010
(86) PCT No.: PCT/JP2010/073502

§ 371 Date: May 22, 2012

(30) Foreign Application Priority Data
Dec. 28, 2009 (JP) 2009-298781

(51) Int. Cl. A61M 37/00

(52) U.S. Cl. 604/24

(57) ABSTRACT

The present invention is to offer an oxygen bath system, which supplies highly concentrated oxygen and a mist of liquid at a predetermined pressure value into a chamber, thereby enabling to efficiently cause oxygen and the mist to be absorbed into the skin and the mucous membrane of a human organism. The oxygen bath system comprises a chamber 51 of accommodating a human organism; a gas supply means 11 of supplying gas containing oxygen of predetermined concentration; a liquid supply means 21 of supplying a liquid; a mist generating means 31 of generating a mist; a pressurizing means 41 of pressurizing the mist and gas containing 25 to 40% oxygen gas generated from gas supplied from the gas supply means 11 and air, and supplying them into the chamber 51; sensors of detecting measured values of air pressure, oxygen concentration, temperature and moisture within the chamber 51; and a control means 71 of controlling the environment within the chamber 51 to be within the ranges of the predetermined set values in response to the measured values detected by the sensors 61, thereby to promoting absorption of the mist and gas into the skin and mucous membrane of the human organism.

### TECHNICAL FIELD

The present invention relates to an oxygen bath system, which supplies highly concentrated oxygen and a mist of liquid at a predetermined pressure value into a chamber accommodating a human organism therein, and causes highly concentrated oxygen and the mist to be absorbed into the skin and the mucous membrane of the human organism.

### BACKGROUND ART

Oxygen existing in the human organism has two kinds of “combination type oxygen” and “solution type oxygen”, and “combination type oxygen” is carried into the human organism linking with hemoglobin in blood due to ordinary pulmonary respiration, while “solution type oxygen” is carried into the human organism as it is solved in blood or body fluids. Solution type oxygen has smaller in size than combination type oxygen, and can pass through blood capillaries, but exists just a little in the human organism under an ordinary life circumstance.

On the other hand, if oxygen of higher concentration than in the atmosphere (about 21%) is supplied into the human organism under the atmosphere of higher pressure than the atmospheric pressure and of highly concentrated oxygen, it is possible to more take oxygen gas as solution type oxygen into the human organism. If solution type oxygen increases in blood or body fluids, it goes around until peripheral organs and improves metabolism and activity of the cell, and further displays disinfection or sterilization effects by oxidation of hyperbaric oxygen. Therefore, this fact has been known in fatigue recovery or health promotion such as earlier curing of injury in the skin, diet or cosmetic acceleration effect for age registration.

As devices supplying highly concentrated oxygen into the human organism under high pressure as mentioned above, a high air pressure system supplying device of highly concentrated oxygen has been now on sale and served (refer to, for example, Patent Documents 1 and 2). This device supplies highly concentrated oxygen at a predetermined pressure value into a chamber for supporting the human organism, and causes highly concentrated oxygen to be absorbed into the skin and mucous membrane of the human organism.

Further, such an oxygen bath system has been also known which accommodates, like a mist sauna, parts of the human organism in its closed space and shower-sprays hot water to the human organism (excepting head region and legs) from an oxygen nozzle (refer to, for example, Patent Documents 3 and 4).

### CITATION LIST

### Patent Documents

Patent Document 1: Japanese Patent Application Publication No. 2004-275706

Patent Document 2: Japanese Patent Application Publication No. 2006-158593

Patent Document 3: Japanese Patent Application Publication No. 2006-75482

Patent Document 4: Japanese Patent Application Publication No. 2008-5916

### SUMMARY OF INVENTION

### Problems to be solved by the Invention

However, these existing oxygen bath devices cause the human organism to be merely bathed with high pressure oxygen of around 1.1 to 1.3 air pressure (in the cases of Patent Documents 1 and 2), otherwise, spray hot water in fog made by oxygen under atmospheric pressure for showering bodies (in the cases of Patent Documents 3 and 4). Considering that highly concentrated oxygen of high air pressure is absorbed into the respiratory organs, in the above cases, oxygen concentration is limited to around 35%. Further on, in relation with the internal ear pressure, air pressure enabling to be applied to the human organism including the head region is limited to around 1.4 air pressure. Under these restrictions, certain limits have been determined for oxygen penetration to the human organism.

In the joint researches with a university's institution, an inventor of this invention has known that if mixing the mist of very fine diameter into highly concentrated oxygen under high pressure and bathing it to the human organism, oxygen absorption into the skin and mucous membrane of the human organism could be rapidly improved. For example, the experiment has proved that in case adding the mist of the fine diameter to the atmosphere of 30% oxygen concentration and 1.25 air pressure, oxygen absorbing efficiency was improved about 30%. It is assumed that if the mist is contacted to the human organism under such a condition of the mist dissolved in or attached to oxygen, the mist penetrates into pores of the skin and the mucous membrane of the human organism. Accordingly, if adding the mist of the fine diameter to air of high pressure of highly concentrated oxygen, oxygen absorbency to the human organism can be improved.

In consideration of the above mentioned prior problems, the present invention is to offer an oxygen bath system, which supplies highly concentrated oxygen and a mist of liquid at a predetermined pressure value into a chamber accommodating the human organism, thereby to cause highly concentrated oxygen and the mist to be absorbed into the skin and the mucous membrane of the human organism.

### Means for Solving the Problems

For settling the above mentioned problems, the present invention is to provide an oxygen bath system, which comprises a chamber of accommodating a human organism; a gas supply means of supplying gas containing oxygen of predetermined concentration; a liquid supply means of supplying a liquid; a mist generating means of generating a mist prepared by pulverizing and dissolving the liquid supplied from the liquid supply means by using gas supplied from the gas supply means; a pressurizing means of pressurizing the mist supplied from the mist generating means and gas containing 25 to 40% oxygen gas generated from gas supplied from the gas supply means and air, and supplying them into the chamber; sensors of detecting measured values of air pressure, oxygen concentration, temperature and moisture within the chamber; and a control means of controlling the environment within the chamber to be within the ranges of the predetermined set values in response to the measured values detected by the sensors, and which is characterized in that the mist prepared by pulverizing and dissolving the liquid and gas containing oxygen gas are supplied under a pressurized condition into the chamber, thereby to promoting absorption of the mist and gas into the skin and mucous membrane of the human organism.

Further, for settling the above mentioned problems, the present invention is to offer an oxygen bath system which comprises a chamber for accommodating a human organism; a gas supply means of supplying gas containing oxygen of predetermined concentration; a liquid supply means of supplying a liquid; a mist generating means of generating a mist prepared by pulverizing and dissolving the liquid supplied from the liquid supply means by using gas supplied from the gas supply means, and supplying the mist into the chamber; a pressuring and supplying means of pressurizing gas containing 25 to 40% oxygen gas generated from gas containing oxygen supplied from the gas supply means and air, and supplying them into the chamber; sensors of detecting measured values of air pressure, oxygen concentration, temperature and moisture in the chamber; and control means of controlling the environment in the chamber to be within the ranges of the predetermined set values in response to the measured values detected by the sensors, and which is characterized in that the mist prepared by pulverizing and dissolving the liquid and gas containing oxygen gas are supplied under pressurization into the chamber, thereby to promoting absorption of the mist and gas into the skin and mucous membrane of the human organism.

By the way, the mist generating means may be disposed in the chamber.

Herein, the chamber of this invention is structured with an acrylic or a glass material, and can withstand against internal pressure of at least 1.5 air pressure.

The gas supply means is composed of a first gas supply means of supplying first gas and a second gas supply means of supplying second gas. Such a structure is sufficient that the mist generating means generates the mist by pulverizing and dissolving the liquid supplied from the liquid supply means with first gas from the first gas supply means, and supplies it to the chamber, and the pressuring means supplies under pressure second gas supplied from the second gas supply means to the chamber.

Incidentally, the chamber is provided with a discharge valve for discharging air and the mist in the chamber in response to increase of air containing oxygen of a predetermined concentration supplied into the chamber and the mist. The sensors include a sensor of detecting the concentration of carbon dioxide, and the control means can also control carbon dioxide in the chamber to be 0.03 to 1% by, for example, adjusting gas displacement in the chamber from the discharge valve and an air content to be supplied.

On the other hand, the liquid has sufficiently any one of or plural combinations with water, ionic water, purified water or sterilized and purified water. The liquids may contain medicines of a respiratory system. Such liquids can contain one or plural aromatic components. As the aromatic components, there may be enumerated essential oils of flowers having effects of calming down human minds (e.g., rose, lavender, rosemary, chamomile, or the like), or aromatic trees (e.g., sandal wood or agarwood).

The mist generated by the mist generating means has preferably diameter sizes of 10 μm or smaller. The control means suitably maintains pressure to be 1.10 to 1.35 air pressure in the chamber. The chamber has a safety valve for discharging gas therein, in case air pressure therein exceeds a predetermined upper limit.

### Advantageous Effects of Invention

According to the present invention, by supplying the mist of liquid together with highly concentrated oxygen into the chamber accommodating the human organism at the predetermined pressure value, oxygen and the mist can be absorbed into the skin and the mucous membrane of the human organism, and efficiency of the oxygen bath system can be improved.

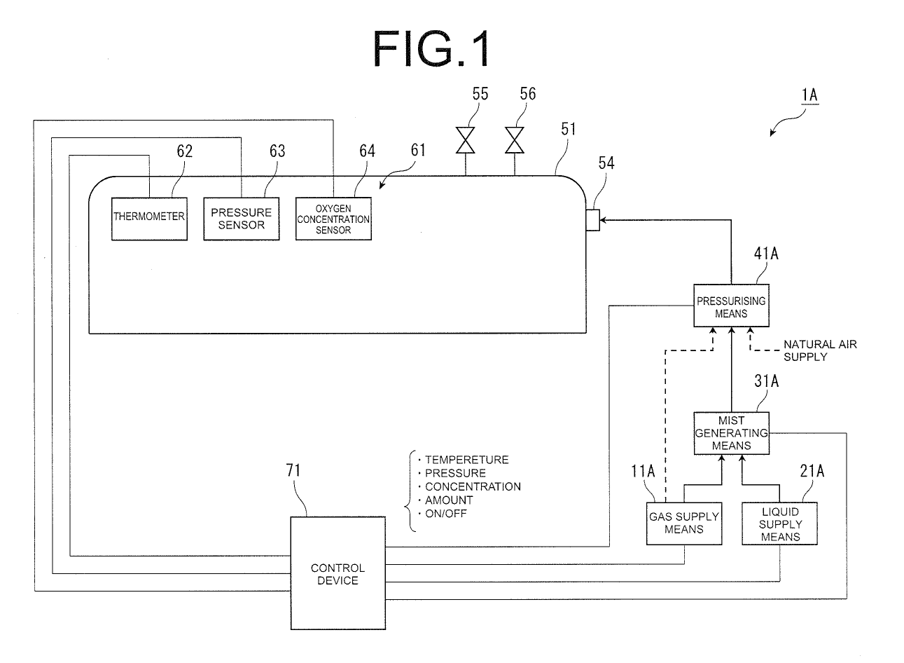

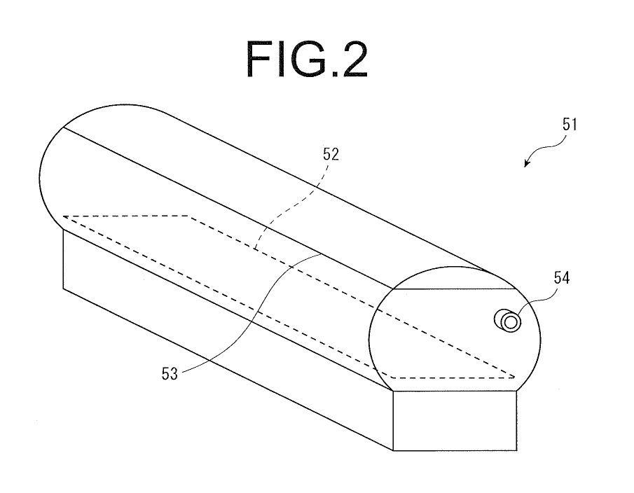

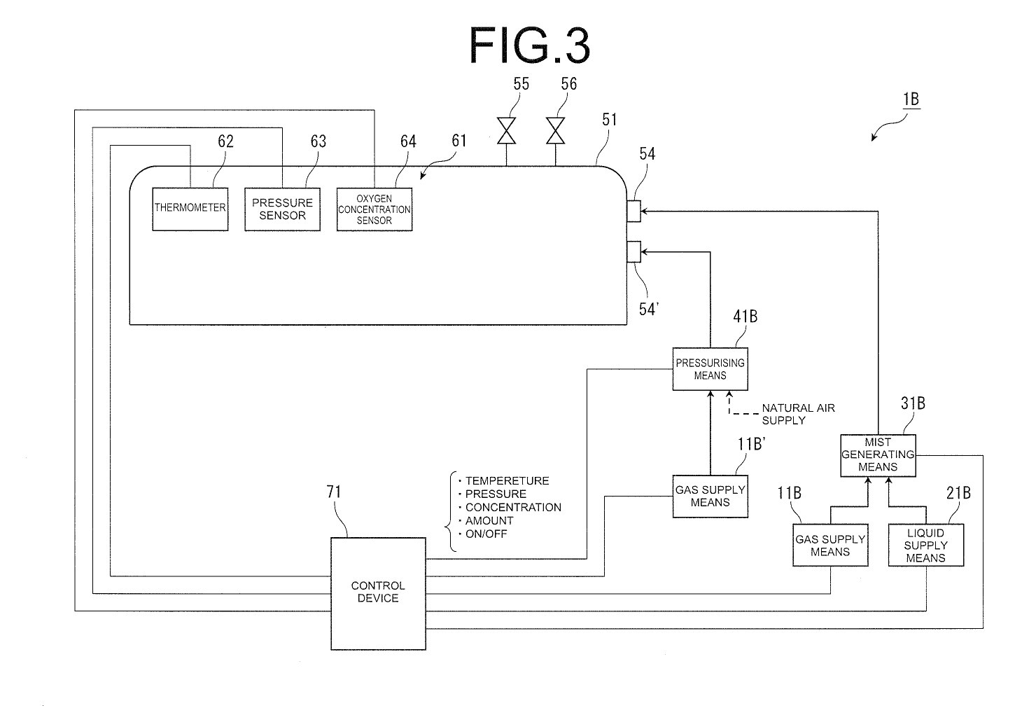

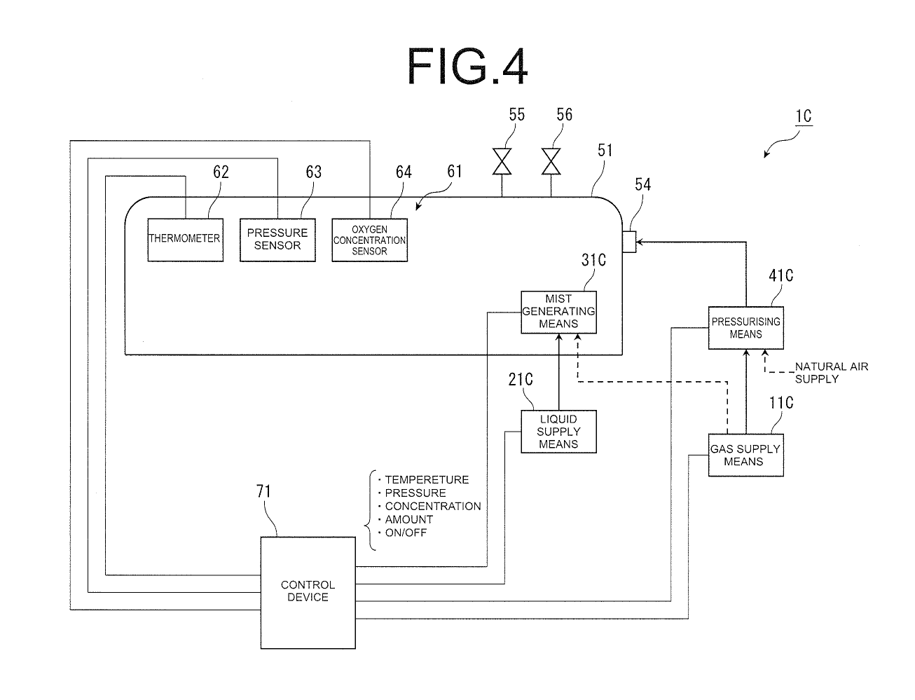

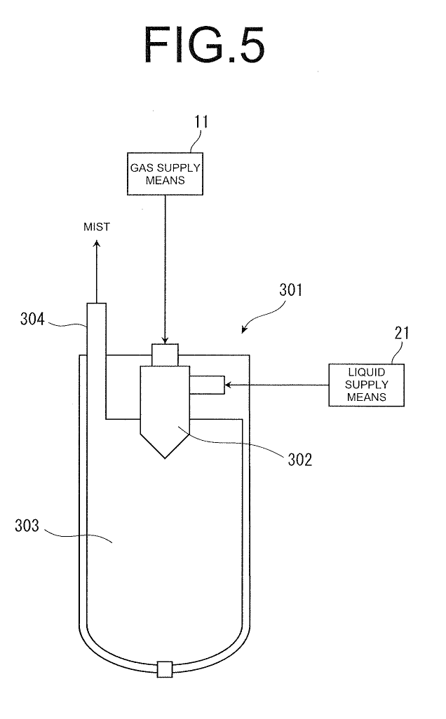

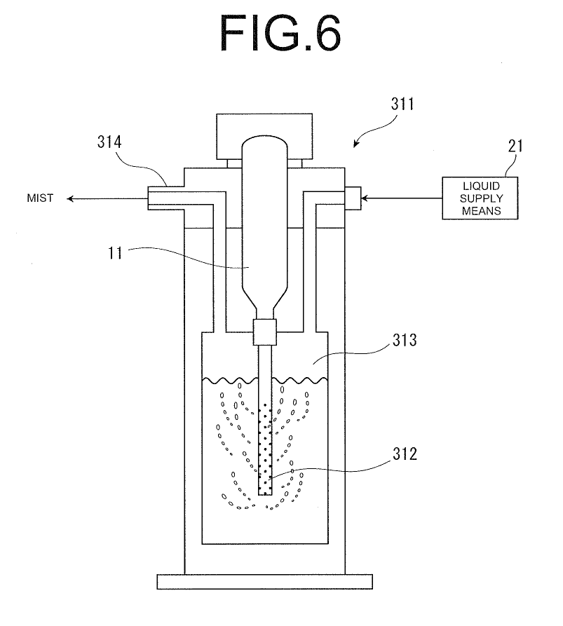

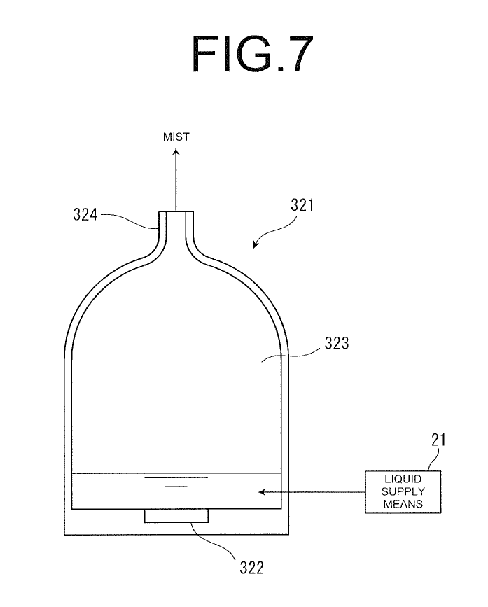

### BRIEF DESCRIPTION OF DRAWINGS

[FIG. 1] A generally schematic view of the oxygen bath system according to a first embodiment of the present invention;

[FIG. 2] A typical view showing one example of the chamber to be applied to the oxygen bath system according to the invention;

[FIG. 3] A generally schematic view of the oxygen bath system according to a second embodiment of the invention;

[FIG. 4] A generally schematic view of the oxygen bath system according to a third embodiment of the invention;

[FIG. 5] A typical view showing one example of a mist generating means to be applied to the oxygen bath system according to the invention (No. 1);

[FIG. 6] A typical view showing one example of the mist generating means to be applied to the oxygen bath system according to the invention (No. 2); and

[FIG. 7] A typical view showing one example of the mist generating means to be applied to the oxygen bath system according to the invention (No. 3).

### DESCRIPTION OF EMBODIMENTS

In the following, with respect to the embodiments of the invention, detailed explanations will be made to, referring to the drawings.

### First Embodiment

FIG. 1 is the generally schematic view of the oxygen bath system according to the first embodiment of this invention. As shown in this Figure, the oxygen bath system 1A comprises a gas supply means 11A of supplying gas containing oxygen at an optional concentration (called appropriately as “gas” in the following) into a mist generating means 31A or a pressurizing means 41A, a liquid supply means 21A of supplying the liquid into the mist generating means 31A, the mist generating means 31A of generating the mist from supplied gas and liquid, the pressurizing means 41A of pressurizing the mist generated in the liquid supply means 31A and supplying into the chamber 51, the chamber 51 of accommodating the human organism, sensors 61 of monitoring the inside of the chamber 51, and a control device 71 of carrying out environmental controls within the chamber 51. The chamber 51 of the invention is structured with acrylic or glass materials, and can withstand against internal pressure of at least 1.5 air pressure.

The gas supply means 11A supplies gas containing oxygen of an optional concentration into the mist generating means 31A, and, for example, an oxygen bomb or an oxygen thickener is suitable. In case the mist is enough supplied in the chamber 51 and concurrently oxygen is short therein, gas may be supplied via the pressurizing means 41A or directly to the chamber 51. The gas supply means 11A is equipped with a regulator for controlling pressure, though not showing. Further, there may be disposed a thermometer (not shown) for controlling temperature, or a heater (not shown) for heating gas.

The liquid supply means 21A is composed of a pump or the like for supplying liquid into the mist generating means 31A. As the liquid, it is preferable to use water, ionic water, purified water or sterilized and purified water. In addition, these liquids may contain medicines effective to users' diseases or their conditions. For example, it may be assumed to contain medicines for the diseases in the respiratory organ system. Such liquids can contain one or plural aromatic components. As the aromatic components, there may be enumerated essential oils of flowers having effects of calming down human minds (e.g., rose, lavender, rosemary, chamomile, or the like), or aromatic trees (e.g., sandal wood or agarwood).

Desirably, this liquid supply means 21A is disposed with a thermometer (not shown) for controlling temperature, or a heater (not shown) for heating the humors.

The mist generating means 31A generates the mist by pulverizing and dissolving gas supplied from gas supply means 11A and the liquid supplied from the liquid supply means 21A, (herein, “pulverizing and dissolving” mean to pulverize the liquid into fine liquid drops and cause them to contact and mix gas). The mist then generated has preferably diameter sizes of 10 μm or smaller.

As the mist generating means 31A, an existing device of producing the mist is applicable. For example, such a device using a fluid nozzle (refer to FIG. 5 later mentioned) is preferable. Otherwise, it is possible to employ a device generating the mist by jetting gas into the fluid at high pressure (refer to FIG. 6), or a device generating the mist by using supersonic wave (refer to FIG. 7).

Herein, a brief explanation will be made to examples of the mist generating means employed in the present invention.

FIG. 5 shows one example of the mist generating means 301 using a fluid nozzle. This mist generating means 301 has a fluid nozzle 302 generating the mist by using a high speed fluid of gas supplied from the gas supply means 11, a storage 303 storing the mist and a mist discharging port 304 discharging the generated mist.

The fluid nozzle 302 produces the mist on a principle of an atomizer or a sprayer, including from those having various structures used to a jet system nebulizer until those enabling to change plural fluids into mists. This invention has no problem of applying any kind of fluid nozzles 302. Further, this mist generating means 301 has a plurality of fluid nozzles 302, and allows such a device of colliding the mists with one another to change liquid drops into minute ones.

FIG. 6 shows one example of a mist generating means 311 of generating the mist by discharging gas into the stored liquid at high pressure. This mist generating means 311 has a gas outlet 312 of discharging gas from the gas supply means 11, a storage 313 of storing the liquid supplied from the liquid supply means 21 and the generated mist, and a mist discharging port 314 of discharging the generated mist. Under a condition that the liquid is stored in the storage 313, if discharging gas from the gas supply means 11 at high pressure, it is possible to change the liquid into a mist. By the way, it is shown that the gas supply means 11 is built-in the mist generating means 311, but an outside-mounting is also enough.

Next, FIG. 7 shows one example of a mist generating means 321 of generating the mist by a supersonic oscillation. This mist generating means 321 has a supersonic oscillator 322 of effecting supersonic oscillation to the liquid supplied from the liquid supply means 21, a storage 323 of storing the liquid supplied from the liquid supply means 21 and the generated mist, and a mist discharging port 324 of discharging the generated mist.

The supersonic oscillator 322 has a piezoelectric element, and if working the piezoelectric element under a condition that the liquid is stored in the storage 323, the supersonic vibration is transmitted over the liquid, and concurrently fine liquid drops occur from the liquid surface and turn out the mist.

By the way, herein, such an example is shown that vibration is given to the liquid supplied from the liquid supply means 21, and a medium of transmitting a supersonic wave may be further furnished.

A pressurizing means 41A is such a means of pressurizing the mist generated in the mist generating means 31A and supplies it to a chamber 51, and for example, preferably, a compressor is employed. This pressurizing means 41A can also pressurize air by natural air supply for controlling the oxygen concentration in the chamber.

The chamber 51 is a container for accommodating the human organism therein, and making a circumstance of oxygen and the mist at more than predetermined concentration exist at predetermined pressure. Materials not deformed at air pressure until at least 1.5 air pressure are served as main materials, which are composed of the resin materials as acryl, glass or reinforced plastic, or metals as aluminum.

FIG. 2 shows one example of the chamber 51. As shown, the chamber 51 has inside a bed plate 52 placing the human organism, an open-close part 53 open and closing the chamber 51, and a supply port 54 of introducing the mist and/or gas. The chamber 51 is sealed for keeping air-tight, and on the other hand, it has a safety valve 55 of automatically discharging air in case an inside air pressure exceeds a limiting value (see FIG. 1). A discharge valve 56 is also furnished for air discharging when necessary (see FIG. 1). By this discharge valve 56, in response to increase of air and the mist containing oxygen of a predetermined concentration supplied into the chamber 51, air and the mist within the chamber are discharged.

Within the chamber 51, several kinds of sensors 61 are disposed for monitoring internal environments. Concretely, those are a thermometer 62, a pressure sensor 63 and an oxygen concentration sensor 64. Although omitting illustrations, these sensors 61 may include a sensor of detecting concentration of carbon dioxide (CO₂) or an air humidity indicator. Thereby, a later mentioned control means 71 is also possible to adjust, for example, the discharging amount of gas in the chamber from the discharge valve, and the amount of air to be supplied, thereby to control the concentration of the carbon dioxide in the chamber to be 0.03 to 1%. For example, when the concentration of carbon dioxide exceeds a determined amount it may be automatically discharged.

The control device 71 is based on the measuring values of these sensors 61 to carry out various kinds of controls for keeping the inside of the chamber 51 under predetermined circumstances. The controls based on the measuring values of the thermometer 62 execute ON-OFF of a heater furnished in the gas supply means 11 and the liquid supply means 21. The controls based on the measuring values of the pressure sensor 63 are discharge air by a discharge valve 56, gas supply or supply stop, mist generation, its supply or supply stop. Further, the controls based on the measuring values of the oxygen concentration sensor 64 are gas supply or supply stop, or air discharge and supply by the discharge valve 56.

The control device 71 is composed of a computer having CPU, memory, and display, and carries out a generation, supply control of the mist and environmental controls within the chamber 51. For example, various kinds of controls are performed such as controls of supplying pressure of oxygen from the gas supply means 11 and temperature, switching ON-OFF of oxygen, switching supply to mist generating means 31/chamber 51, control of supplying pressure of liquid from the liquid supply means 21 or control of temperature, switching ON-OFF of supply, switching ON-OFF of mist supply from the mist generating means 31, or temperature, air pressure, oxygen concentration and mist amount in the chamber 51. Thus, the oxygen bath can be taken, adding the mist under pre-determined conditions. In particular, for checking the dewed mist dropping to be a minimum limit within the chamber, controls of temperature, air pressure and mist amount are carried out. By the way, the present device is structured in such a way that when the pressure value in the chamber 51 exceeds a predetermined value, supply of the mist or gas is stopped by the control device 71.

Herein, standards of proper environments predetermined by the control device 71 within the chamber 51 are (1) air pressure: 1.10 to 1.35, more preferably 1.2 to 1.3, (2) oxygen concentration: not less than 20%, more preferably 30% to 40%, and (3) temperature: 20 to 30° C.

In regard to the mist supply pipe connecting the mist generating means 31 and the chamber 51, in the inside of the mist supply pipe, the shape of a so-called cornice shaped hose is good for supplying only such mists of small diameter into the chamber 51, and further, for obtaining flexibility (easily bending) of the mist supply pipe.

In the meantime, the first embodiment is structured in such a manner that the pressurizing means 41A is disposed in the downstream side of the mist generating means 31A and pressurizes the generated mist together with natural air suction, but reversely, such a structure is also sufficient that the pressurizing means 41A is disposed in the upstream side of the mist generating means 31A, connecting the pressurizing means 41A to the gas supply means 11A together with the mist generating means 31A to generate a pressurized mist by using pressurized gas, using natural air suction by controlling the control device 71.

In this embodiment, the mist generated by the mist generating means 31 is mixed into gas containing oxygen at an optional concentration, and is supplied into the chamber 51 under pressure by the pressurizing means 41A. The pressurizing means 41A can send, into the chamber 51, only gas containing oxygen by natural air suction or of the optional concentration by the environment prevailing in the chamber 51.

By structuring in such a way, absorption of oxygen into the human organism can be accelerated. In a case of containing medicines in the mist, the medicine can further add its effect.

### Second Embodiment

Next, the present invention will be explained in regard to a second embodiment. In the embodiment, explanation will be made to supply of the mist and the oxygen bath system 1B of such a structure carrying out pressurization within the chamber via a different system. The same parts as those of the first embodiment shown in FIG. 1 will be given the same numerals, omitting detailed explanation. Especially, in the following, excepting that difference is in the structure of the supplying means (gas supply means 11, liquid supply means 21, mist generating means 31 and pressurizing means 41) of the mist and gas into the chamber, the structure is the same as that of FIG. 1.

FIG. 3 is the generally schematic view of the oxygen bath system according to the second embodiment of this invention. As shown in this Figure, the oxygen bath system 1B of this embodiment comprises a gas supply means 11B of supplying gas containing oxygen at an optional concentration into a mist generating means 31B, a liquid supply means 21B of supplying the liquid into the mist generating means 31B, the mist generating means 31B of generating the mist from supplied gas and liquid, a gas supply means 11B′ of supplying gas containing oxygen at an optional concentration into a pressurizing means 41B, the pressurizing means 41B of pressurizing gas supplied from the gas supply means 11B′ and supplying into the chamber 51, the chamber 51 of accommodating the human organism, the sensors 61 of monitoring the inside of the chamber 51, and the control device 71 of carrying out environmental controls within the chamber 51.

The gas supply means 11B supplies gas containing oxygen at the optional concentration into the mist generating means 31B, and, for example, an oxygen bomb or an oxygen thickener is appropriate.

On the other hand, the gas supply means 11B′ supplies gas containing oxygen at the optional concentration into the pressurizing means 41B. It is sufficient to supply gas containing oxygen at the same concentration as that of the gas supply means 11B, or different concentration is enough. For example, the oxygen bomb or the oxygen thickener is appropriate.

These gas supply means 11B, 11B′ are provided with regulators for controlling pressure, though not showing. Further, a thermometer (not shown) for controlling temperature, and a heater (not shown) for increasing temperature are sufficient to be furnished.

The liquid supply means 21B is made of a pump or the like, supplying the liquid to the mist generating means 31B. The liquid to be supplied herein is the same as that of the first embodiment. Desirably, the liquid supply means 21B is provided with the thermometer (not shown) for controlling temperature, and the heater (not shown) for increasing temperature.

The mist generating means 31B generates the mist prepared by pulverizing and dissolving gas which is supplied from the gas supply means 11B and the liquid which is supplied from the liquid supply means 21B, (herein, “pulverizing and dissolving” mean to pulverize the liquid into fine liquid drops and cause them to contact and mix gas). The mist then generated has preferably diameter sizes of 10 μm or smaller. As the mist generating means 31B, an existing device of producing the mist is applicable. For example, such a device using the fluid nozzle (refer to FIG. 5) is preferable. Otherwise, it is possible to employ a device generating the mist by jetting gas into the fluid at high pressure (refer to FIG. 6), or a device is suitable which is able to pressurize the mist and supply it.

A pressurizing means 41B is such a means of pressurizing gas supplied from the gas supply means 11B′ and supplies it to the chamber 51, and for example, preferably, a compressor is employed. This pressurizing means 41B can also pressurize air having been effected with natural air supply for controlling the oxygen concentration in the chamber 51.

In the present embodiment, gas containing oxygen at an optional concentration is mixed with the mist generated by the mist generating means 31 and supplied into the chamber 51, and further gas is supplied under pressure into the chamber 51 by the pressurizing means 41B. The pressurizing means 41B can send to the chamber 51 only such gas containing oxygen by natural air supply or at an optional concentration depending on the environment in the chamber 51 by controlling the control device 71.

Similarly in the first embodiment, in regard to the mist generating means 31 and the mist supply pipe connecting a pipe of the chamber 51, the shape of so-called cornice shaped hose is good for supplying only such mists of small diameter into the chamber 51 in the inside of the mist supply pipe, and further, for providing flexibility (easily bending) of the mist supply pipe.

By structuring in such a way, absorption of oxygen into the human organism can be accelerated. In a case of containing medicine in the mist, the medicine can further add its effect.

### Third Embodiment

Next, the present invention will be explained in regard to a third embodiment. In this embodiment, explanation will be made to the oxygen bath system 1C of a structure having the mist generating means inside the chamber. The same parts as those of the first embodiment shown in FIG. 1 will be given the same numerals, omitting detailed explanations. Especially, in the following, excepting that difference is in the structure of the supplying means (gas supply means 11, liquid supply means 21, mist generating means 31 and pressurizing means 41) of the mist and gas into the chamber, the structure is the same as that of FIG. 1.

FIG. 4 is the generally schematic view of the oxygen bath system according to the third embodiment of this invention. As shown in this Figure, the oxygen bath system 1C comprises the gas supply means 11C of supplying gas containing oxygen at an optional concentration in a pressurizing means 41C, the liquid supply means 21C of supplying the liquid into the mist generating means 31C, the mist generating means 31C of generating the mist from the supplied liquid, the pressurizing means 41C of pressurizing gas supplied from the gas supply means 11C and supplying it into the chamber 51, the chamber 51 of accommodating the human organism, the sensors 61 of monitoring the inside of the chamber 51, and the control device 71 of carrying out environmental controls within the chamber 51.

The gas supply means 11C supplies gas containing oxygen at the optional concentration into the pressurizing means 41C, and, for example, an oxygen bomb or an oxygen thickener is appropriate. This gas supply means 11C is furnished with a regulator for controlling pressure, though not showing. Further, a thermometer (not shown) for controlling temperature, and a heater (not shown) for increasing temperature are sufficient.

The liquid supply means 21C is made of a pump or the like, supplying the liquid to the mist generating means 31C. The liquid to be supplied herein is the same as that of the first embodiment. Desirably, the liquid supply means 21C is provided with the thermometer (not shown) for controlling temperature, and the heater (not shown) for increasing temperature. By the way, the liquid supply means 21C is shown as being provided outside of the chamber 51, and may be provided inside of the chamber 51.

The mist generating means 31C is a device of generating the mist from the liquid supplied from the liquid supply means 21C. The mist then generated has preferably diameter sizes of 10 μm or smaller. As the mist generating means 31C, an existing device of producing the mist is applicable. A device enabling to generate the mist, not using a gas flow, in particular, the device generating the mist by using supersonic wave (refer to FIG. 7) is suitable, and in a case of using the gas flow, as showing with a dotted line in FIG. 4, gas is supplied from the gas supply means 11C. Attention should be paid to that the mist generating means 31C must be a pressure resistant device, since it is placed within the chamber 51.

A pressurizing means 41C is such a means of pressurizing gas supplied from the gas supply means 11C and supplies it to the chamber 51, and for example, preferably, a compressor is employed. Further, this pressurizing means 41C can also pressurize air by natural air supply for controlling the oxygen concentration in the chamber 51.

The present embodiment supplies under pressure gas containing oxygen at an appropriate concentration into the chamber 51 by the pressurizing means 41C, and at the same time, generates the mist in the chamber 51 by the mist generating means 31C. The pressurizing means 41C can send to the chamber 51 only such gas containing oxygen by natural air supply or at an optional concentration depending on the environment within the chamber 51 by controlling the control device 71.

By structuring in such a way, absorption of oxygen into the human organism can be accelerated. Ina case of containing medicine in the mist, the medicine can further add its effect.

As mentioned above, depending on the oxygen bath system of this invention, within the chamber accommodating the human organism, oxygen of high concentration and the liquid mist can be supplied to at a desired pressuring value, so that oxygen and the mist can be efficiently absorbed into the skin and mucous membrane of the human organism, and the effect of the oxygen bath can be heightened.

In the above mentioned, the embodiments of the invention have been explained, and the invention is not limited to the above mentioned embodiments, and various modifications can be provided based on the subject matters of the invention and are not excluded from the scope of the invention.

### INDUSTRIAL APPLICABILITY

The present invention relates to the oxygen bath system, which supplies highly concentrated oxygen and the mist of liquid at a predetermined pressure value into the chamber supporting a human organism therein, and causes highly concentrated oxygen and the mist to be absorbed into the skin and the mucous membrane of the human organism, having industrial applicability.

### REFERENCE SIGNS LIST

1A, 1B, 1C: oxygen bath system

11A, 11B, 11B′, 11C: gas supply means

21A, 21B, 21C: liquid supply means

31A, 31B, 31C: mist generating means

301, 311, 321: mist generating means

302: fluid nozzle

303, 313, 323: storage

304, 314, 324: mist discharging port

312: gas outlet

322: supersonic oscillator

41A, 41B, 41C: pressurizing means

51: chamber

52: bed plate

53: open-close part

54: supply port

55: safety valve

56: discharge valve

61: sensor

62: thermometer

63: pressure sensor

64: oxygen concentration sensor

71: control device

1. An oxygen bath system, comprising
a chamber of accommodating a human organism;
a gas supply means of supplying gas containing oxygen of predetermined concentration;
a liquid supply means of supplying a liquid;
a mist generating means of generating a mist prepared by pulverizing and dissolving the liquid supplied from the liquid supply means by using gas supplied from the gas supply means;
a pressurizing means of pressurizing the mist supplied from the mist generating means and gas containing 25 to 40% oxygen gas generated from gas supplied from the gas supply means and air, and supplying them into the chamber;
sensors of detecting measured values of air pressure, oxygen concentration, temperature and moisture within the chamber; and
a control means of controlling the environment within the chamber to be within the ranges of the predetermined set values in response to the measured values detected by the sensors,
wherein the mist prepared by pulverizing and dissolving the liquid and gas containing oxygen gas are supplied under a pressurized condition into the chamber, thereby to promoting absorption of the mist and gas into the skin and mucous membrane of the human organism.

2. An oxygen bath system, comprising
a chamber for accommodating a human organism;
a gas supply means of supplying gas containing oxygen of predetermined concentration;
a liquid supply means for supplying a liquid;
a mist generating means of generating a mist prepared by pulverizing and dissolving the liquid supplied from the liquid supply means by using gas supplied from the gas supply means, and supplying the mist into the chamber;
a pressurizing and supplying means of pressurizing gas containing 25 to 40% oxygen gas generated from gas containing oxygen supplied from the gas supply means and air, and supplying them into the chamber;
sensors of detecting measured values of air pressure, oxygen concentration, temperature and moisture in the chamber, and control means of controlling the environment in the chamber to be within the ranges of the predetermined set values in response to the measured values detected by the sensors,
wherein the mist prepared by pulverizing and dissolving the liquid and gas containing oxygen gas are supplied under pressurization into the chamber, thereby to promoting absorption of the mist and gas into the skin and mucous membrane of the human organism.

3. The oxygen bath system as set forth in claim 2, wherein the mist generating means is disposed in the chamber.

4. The oxygen bath system as set forth in claim 1, wherein the chamber is structured with an acrylic or a glass material, and can withstand against internal pressure of at least 1.5 air pressure.

5. The oxygen bath system as set forth in claim 2, wherein
the gas supply means is composed of a first gas supply means of supplying first gas and a second gas supply means of supplying second gas,
the mist generating means generates the mist by pulverizing and dissolving the liquid supplied from the liquid supply means with first gas from the first gas supply means, and supplies it to the chamber, and
the pressuring means supplies under pressure second gas supplied from the second gas supply means to the chamber.

6. The oxygen bath system as set forth in claim 1, wherein the chamber is provided with a discharge valve of discharging air and the mist in the chamber in response to increase of air containing oxygen of a predetermined concentration supplied into the chamber and the mist.

7. The oxygen bath system as set forth in claim 6, wherein sensors include a sensor of detecting the concentration of carbon dioxide, and
the control means can also control carbon dioxide in the chamber to be 0.03 to 1%

8. The oxygen bath system as set forth in claim 1, wherein the liquid has sufficiently any one or plural combination with water, ionic water, purified water or sterilized and purified water.

9. The oxygen bath system as set forth in claim 8, wherein the liquids can contain one or plural aromatic components.

10. The oxygen bath system as set forth in claim 1, wherein the mist generated by the mist generating means has preferably diameter sizes of 10 μm or smaller.

11. The oxygen bath system as set forth in claim 1, wherein the controlling means maintains pressure to be 1.10 to 1.35 air pressure in the chamber.

12. The oxygen bath system as set forth in claim 11, wherein the chamber has a safety valve for discharging gas therein, in case air pressure therein exceeds a predetermined upper limit.
